(19) United States
(12) Patent Application Publication
(10) Pub. No.: US 2025/0259748 A1
(43) Pub. Date: Aug. 14, 2025

(54) MENOPAUSE PREDICTING TOOL

(71) Applicant: Timeless Biotech Incorporated, Wilmington, DE (US)

(72) Inventors: Kiran Kumar, La Jolla, CA (US); Jason Bryant, Riverdale, MD (US)

(21) Appl. No.: 19/192,052

(22) Filed: Apr. 28, 2025

Related U.S. Application Data
(63) Continuation-in-part of application No. 19/049,414, filed on Feb. 10, 2025.
(60) Provisional application No. 63/551,705, filed on Feb. 9, 2024.

(51) Int. Cl. G16H 50/20; G16H 10/40; G16H 10/60

(52) U.S. Cl. CPC G16H 50/20; G16H 10/40; G16H 10/60

(57) ABSTRACT

An Artificial Intelligence (AI) system trained by a plurality of training data. The AI system is trained by relationships between a time to final menstrual period and ratios between follicle-stimulating hormone levels and estradiol levels, anti-mullerian hormone, race, cholesterol levels, and a presence of one or more contraceptives. The AI system is configured to accept data from only one sample from a user as input and generate a prediction of a time to final menstrual period for the user as output.

### CROSS-REFERENCE TO RELATED APPLICATIONS

This application is a continuation-in-part and claims benefit of U.S. application Ser. No. 19/049,414 filed Feb. 10, 2025, which is a non-provisional and claims benefit of U.S. Provisional Application No. 63/551,705 filed Feb. 9, 2024, the specifications of which are incorporated herein in their entirety by reference.

### FIELD OF THE INVENTION

The present invention is directed to a time-to-menopause predicting algorithm that uses test results from a single blood draw.

### BACKGROUND OF THE INVENTION

Every female who hasn't had her uterus removed before puberty will experience menopause. This transition can be quite severe and catches women off guard. Perimenopause starts up to 10 years before menopause, starting as early as age 35. Approximately 21% of women experience menopause outside the standard range of 44 to 54 years old. Furthermore, on average, African American and Hispanic women have an earlier menopause than caucasian women. Healthcare providers are reluctant to diagnose menopause in these women, resulting in a more difficult transition and the increased likelihood of preventable disease like osteoporosis, heart disease, dementia, etc. Menopause education is rare, even though it is known to aid with a smooth transition.

Delayed menopause diagnosis is quite common and can lead to deteriorating health effects. 31% of women are misdiagnosed, claiming it took multiple appointments with their GP to be properly diagnosed with menopause. 37% of women are wrongly prescribed, usually being prescribed antidepressants rather than HRT. Studies show that one main reason women are struggling with menopause is that there's no test that can tell a woman whether she's entered perimenopause or menopause. Starting HRT on time before the final menstrual period yields several health benefits for the individual and suppresses symptoms. Thus, there exists a present need for a menopause predicting algorithm that uses test results from a single blood draw.

### BRIEF SUMMARY OF THE INVENTION

It is an objective of the present invention to provide systems, methods, and non-transitory computing media that allow for a menopause predicting algorithm that uses test results from a single blood draw, as specified in the independent claims. Embodiments of the invention are given in the dependent claims. Embodiments of the present invention can be freely combined with each other if they are not mutually exclusive.

The present invention features a computer system for predicting a time to a final menstrual period (FMP) in a patient. The system may comprise an artificial intelligence (AI) model, trained by a training data set comprising relationships between a time to final menstrual period and ratios between follicle-stimulating hormone (FSH) levels and estradiol levels, configured to accept FSH data and estradiol levels from a user as input and generate a prediction of a time to final menstrual period as output.

The present invention features an algorithm to predict the time to final menstrual period (FMP) through the use of a blood test and demographic data. The data and systems of the present invention allow for personalization for the ratios and dosage of bioidentical hormones, or non-hormonal options, that can be taken in preparation for menopause and during/post-menopause. This not only assists with the “treatment” of menopause but also assists with the identification and “treatment” of hormonal imbalances.

In some embodiments, the present invention accounts for factors such as race and geographical location to enable greater accuracy. In some embodiments, the present invention implements feature engineering to improve predictive capabilities. The present invention features a flexible system that is configured to integrate new data and variables. In some embodiments, the system of the present invention may take into account variables from cohort data, vaginal microbiome data, or a combination thereof.

One of the unique and inventive technical features of the present invention is the implementation of domain-specific biomarker ratios, specifically the Follicle Stimulating Hormone (FSH) to Estradiol ratio. Without wishing to limit the invention to any theory or mechanism, it is believed that the technical feature of the present invention advantageously provides for the ability to make accurate predictions of the time until a patient's final menstrual cycle based on a single-point-in-time measurement, eliminating the need for repeated longitudinal sampling. None of the presently known prior references or works have the unique inventive technical feature of the present invention.

Furthermore, the inventive technical feature of the present invention contributed to a surprising result. For example, one of ordinary skill in the art would implement individual features well-known in the field to be connected to menopause/perimenopause (e.g., cycle timing, properties over multiple blood tests). The present invention implements prediction of the time until the patient's final menstrual cycle based on the Follicle Stimulating Hormone (FSH) to Estradiol ratio. Surprisingly, this allows for accurate and efficient prediction based on a one-time measurement. Thus, the inventive technical feature of the present invention contributed to a surprising result.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a flow chart of a method for training and developing the AI system of the presently claimed invention.

FIGS. 2A-2B show a flow chart diagram of an example use case of the AI system for menopause prediction of the present invention.

FIG. 3 shows a line graph diagram of the root-mean-square (RMS) error of the AI system for menopause prediction of the present invention.

FIG. 4 shows a flow chart of the method implemented by the AI system for menopause prediction of the present invention.

FIG. 5 shows a flow chart of the patient experience of implementing the method and system for menopause prediction of the present invention.

FIG. 6A shows a schematic diagram of a system for predicting a time to a FMP in a patient of the present invention.

FIG. 6B shows a flowchart of a method for predicting a time to a FMP in a patient of the present invention.

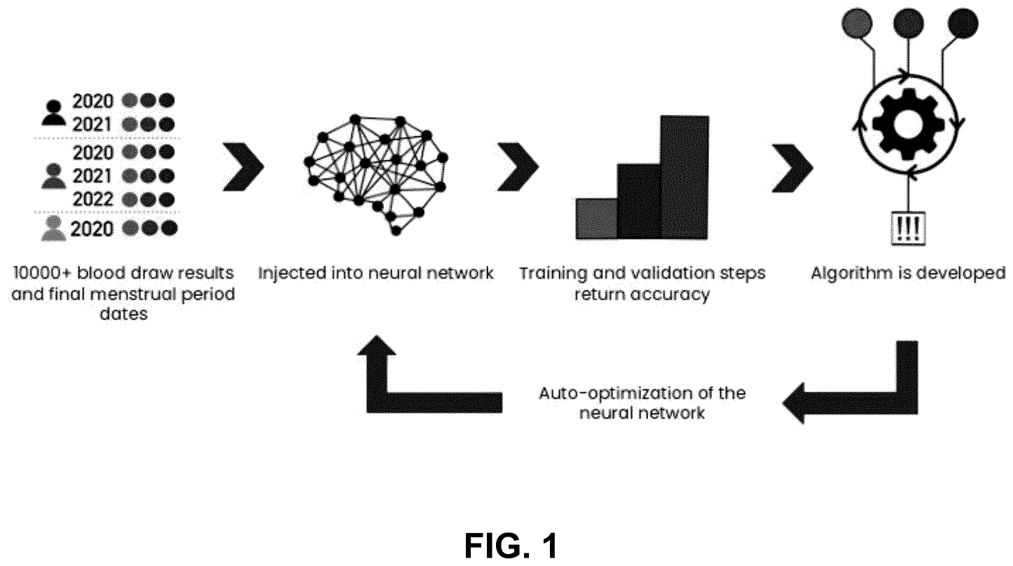

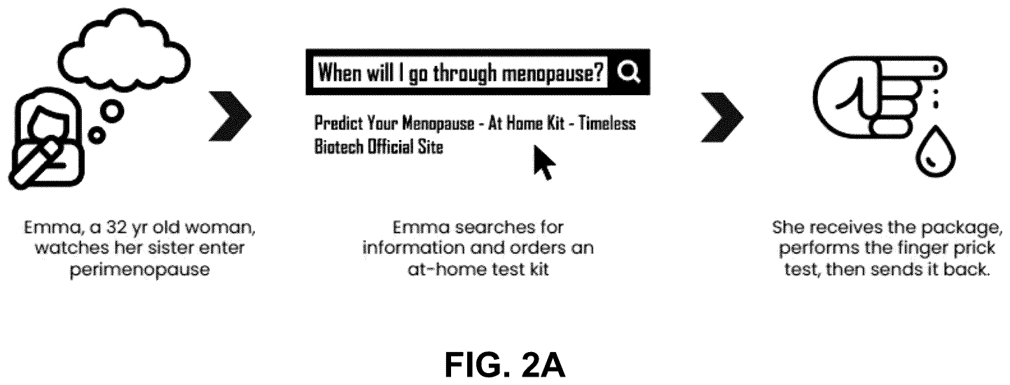

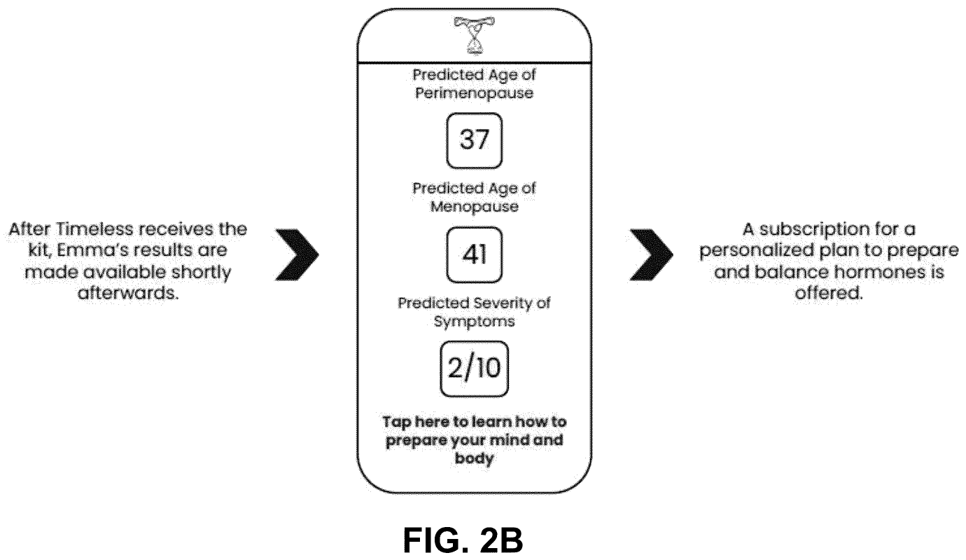

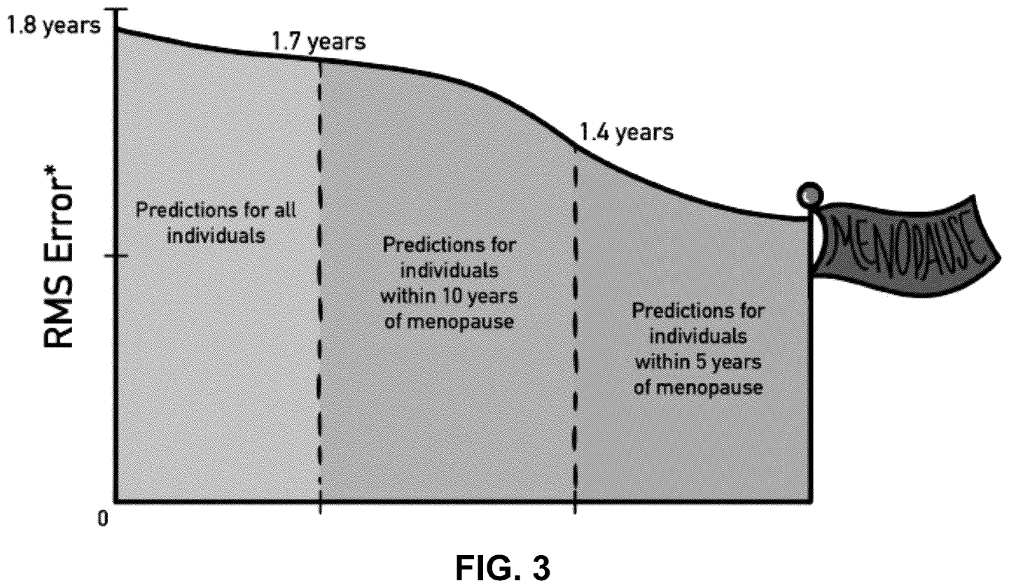

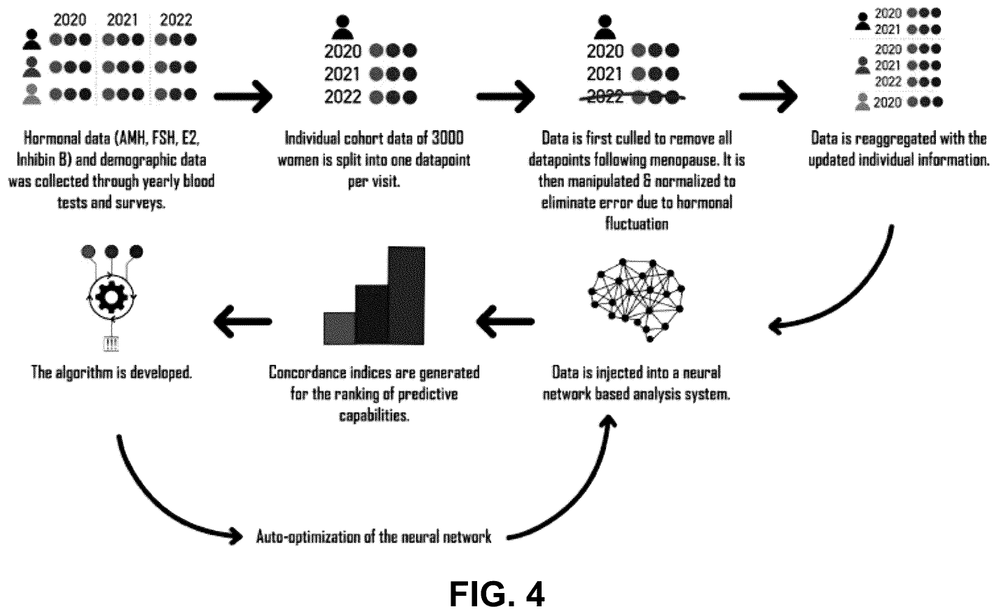

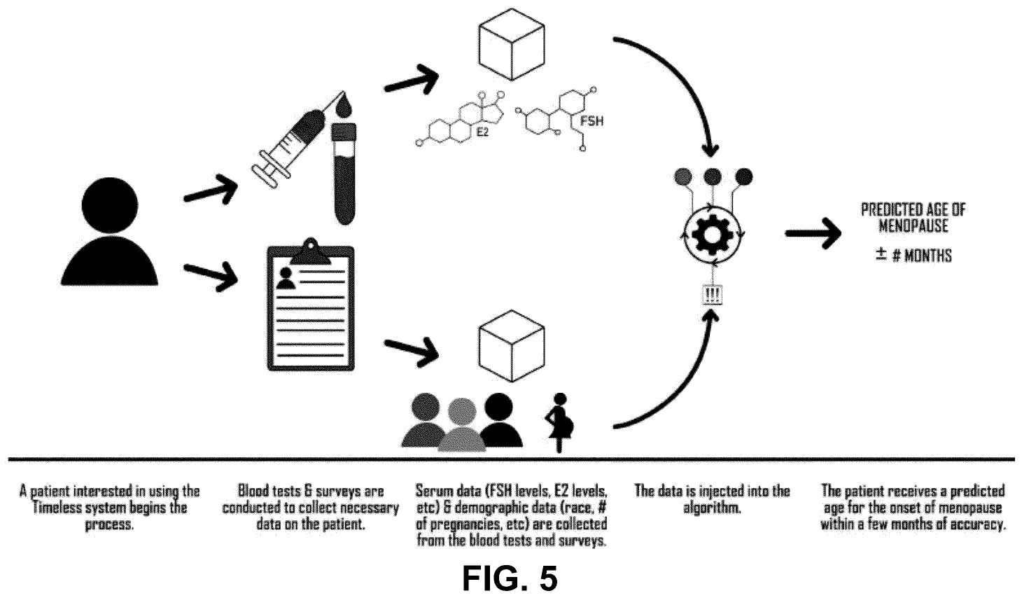

### DETAILED DESCRIPTION OF THE INVENTION

Following is a list of elements corresponding to a particular element referred to herein:
100 system
110 processor
120 memory component
121 artificial intelligence model
200 patient

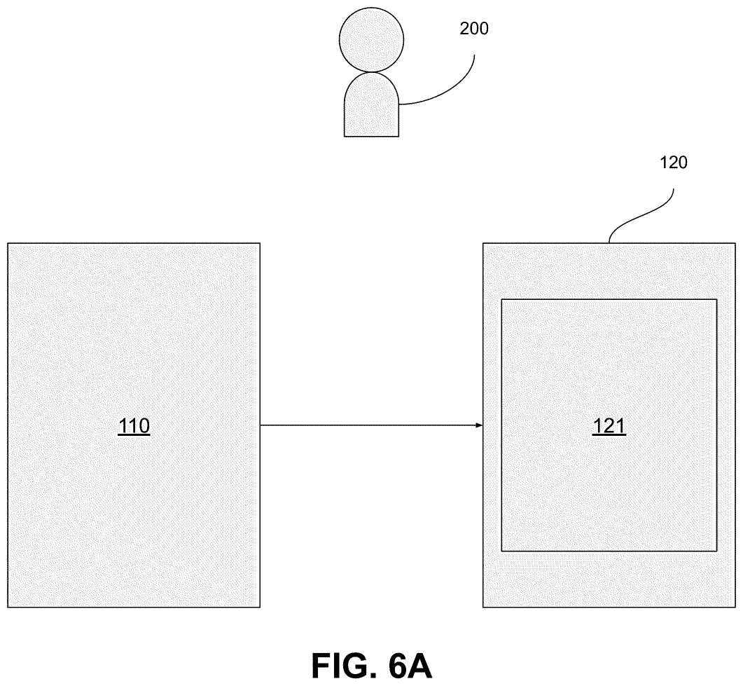

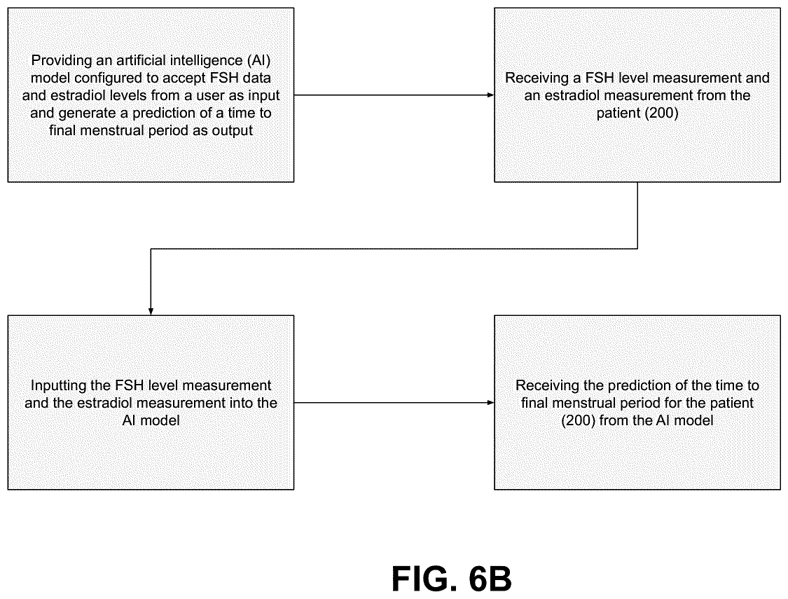

Referring now to FIG. 6A, the present invention features a computer system (100) for predicting a time to a final menstrual period (FMP) in a patient. In some embodiments, the system (100) may comprise a processor (110) configured to execute computer-readable instructions. The system (100) may further comprise a memory component (120) operatively coupled to the processor (110). The memory component (120) may comprise an artificial intelligence (AI) model (121), trained by a training data set comprising relationships between a time to final menstrual period and ratios between follicle-stimulating hormone (FSH) levels and estradiol levels, configured to accept FSH data and estradiol levels from a user as input and generate a prediction of a time to final menstrual period as output.

The memory component (120) may further comprise computer-readable instructions for receiving a FSH level measurement and an estradiol measurement from the patient. The computer-readable instructions may further comprise inputting the FSH level measurement and the estradiol measurement into the AI model (121). The computer-readable instructions may further comprise receiving the prediction of the time to FMP for the patient from the AI model (121).

In some embodiments, the training data set may further comprise relationships between time to FMP and geographical location, age, race, cholesterol, medications, or a combination thereof. The AI model (121) may be further configured to accept geographical location, age, race, cholesterol, medications, or a combination thereof from the patient as input. In some embodiments, the FSH level measurement and the estradiol measurement may be derived from a single blood sample from the patient. In some embodiments, the system (100) may be implemented on a local computer, a cloud computing system, or a combination thereof. In some embodiments, the training data set may be standardized by a z-score normalization process. In some embodiments, the training data set may comprise incomplete data supplemented by a Kaplan-Meier estimation.

Referring again to FIG. 6A, the present invention features a computer system (100) for predicting a time to a FMP in a patient (200). In some embodiments, the system (100) may comprise a processor (110) configured to execute computer-readable instructions. The system (100) may further comprise a memory component (120) operatively coupled to the processor (110). The memory component (120) may comprise an artificial intelligence (AI) model (121), trained by a training data set comprising relationships between a time to FMP and ratios between follicle-stimulating hormone (FSH) levels and estradiol levels, configured to accept FSH data and estradiol levels from a user as input and generate a prediction of a time to FMP as output.

The memory component (120) may further comprise computer-readable instructions for receiving a FSH level measurement and an estradiol measurement from the patient (200). The computer-readable instructions may further comprise receiving menstrual cycle data from the patient (200). The computer-readable instructions may further comprise adjusting the FSH level measurement, the estradiol measurement, or a combination thereof based on the menstrual cycle data. The computer-readable instructions may further comprise inputting the adjusted FSH level measurement and the estradiol measurement into the AI model (121). The computer-readable instructions may further comprise receiving the prediction of the time to FMP for the patient (200) from the AI model (121).

In some embodiments, the training data set may further comprise relationships between time to FMP and geographical location, age, race, cholesterol, medications, or a combination thereof. The AI model (121) may be further configured to accept geographical location, age, race, cholesterol, medications, or a combination thereof from the patient (200) as input. In some embodiments, the FSH level measurement and the estradiol measurement may be derived from a single blood sample from the patient (200). In some embodiments, the system (100) may be implemented on a local computer, a cloud computing system, or a combination thereof. In some embodiments, the training data set may be standardized by a z-score normalization process. In some embodiments, the training data set may comprise incomplete data supplemented by a Kaplan-Meier estimation.

Referring now to FIG. 6B, the present invention features a computer-implemented method for predicting a time to a FMP (FMP) in a patient (200). The method may comprise providing an artificial intelligence (AI) model, trained by a training data set comprising relationships between a time to FMP and ratios between follicle-stimulating hormone (FSH) levels and estradiol levels, configured to accept FSH data and estradiol levels from a user as input and generate a prediction of a time to FMP as output. The method may further comprise receiving a FSH level measurement and an estradiol measurement from the patient (200). The method may further comprise inputting the FSH level measurement and the estradiol measurement into the AI model. The method may further comprise receiving the prediction of the time to FMP for the patient (200) from the AI model.

In some embodiments, the method may further comprise receiving menstrual cycle data from the patient (200) and adjusting the FSH level measurement, the estradiol measurement, or a combination thereof based on the menstrual cycle data.

The method of claim 13, wherein the training data set further comprises relationships between time to FMP and geographical location, age, race, cholesterol, medications, or a combination thereof, wherein the AI model is further configured to accept geographical location, age, race, cholesterol, medications, or a combination thereof from the patient (200) as input. In some embodiments, the method may further comprise receiving a single blood sample from the patient (200) and deriving the FSH level measurement and the estradiol measurement from the single blood sample. In some embodiments, the method may be implemented on a system (100) comprising a local computer, a cloud computing system, or a combination thereof. In some embodiments, the training data set may be standardized by a z-score normalization process. In some embodiments, the training data set may comprise incomplete data supplemented by a Kaplan-Meier estimation.

In some embodiments, the present invention features an Artificial Intelligence (AI) system trained by a plurality of training data comprising relationships between a time to FMP and ratios between follicle-stimulating hormone levels and estradiol levels, race, cholesterol levels, and a presence of one or more contraceptives, configured to accept data from only one sample from a user as input and generate a prediction of a time to FMP for the user as output.

The sample may be collected from an at-home test kit. The sample may comprise a blood sample. The training data may comprise 10000+ blood draw results and final menstrual period dates. The AI model may be auto-optimized.

For final menstrual period dates, the highest weight of predictive values is the ratio between FSH and estradiol. This allows for the use of just one blood reading to make an accurate prediction. In some embodiments, the AI model may additionally accept data on the user's ovarian cycle to increase accuracy. In some embodiments, the training data may comprise data on patients who have not yet had a final menstrual event. To overcome this, a Kaplan-Meier estimator is used to fill in the blanks and estimate the final menstrual period, thus allowing this to still be used as training data.

The AI model of the present invention integrates a modified Cox proportional hazards model with a custom-engineered feedforward neural network, replacing the linear component with a nonlinear risk function trained end-to-end. While this alone may resemble existing deep survival models, the AI model of the present invention introduces a novel domain-specific adaptation: the use of biologically informed feature engineering, most notably the FSH-to-Estradiol ratio, and the inclusion of an individual's position within their menstrual cycle to account for hormonal variability. These adaptations enable precise prediction of time to menopause from a single blood draw, a feature not supported by existing survival models or menopause prediction systems. The invention solves a long-standing problem in clinical and consumer settings: how to produce accurate time-to-menopause estimates with only one blood test despite high individual-to-individual hormone fluctuation.

A key inventive element of the system is its use of domain-specific biomarker ratios, specifically the Follicle Stimulating Hormone (FSH) to Estradiol ratio, rather than raw hormone values. This ratio is a clinically meaningful indicator of reproductive stage and is more stable than absolute values, which are subject to high individual-to-individual and intra-cycle variability. By incorporating this ratio, the model is uniquely capable of making accurate predictions from a single-point-in-time measurement, eliminating the need for repeated longitudinal sampling. Furthermore, the system stores and utilizes an individual's relative menstrual cycle stage, enabling context-aware interpretation of hormone levels-another novel aspect not found in traditional survival models or general-purpose AI health systems.

Individual Layers: The individual layers vary in their structure. Although they follow a format of weighted edges pointing to every node in the subsequent layer, each of which has its own bias term and activation function, the values of the weights and biases, the type of activation function, and the number of nodes per layer vary. The architecture is designed to balance model complexity and generalizability, ensuring accurate risk stratification while minimizing overfitting.

Activation Functions: The primary activation function used is ReLU (Rectified Linear Unit) for its efficiency in training deep networks. Additionally, sigmoid and tanh activations may be incorporated for specific layers where output constraints or feature transformations benefit from bounded activation.

Connections Between Layers: The primary activation function used is Rectified Linear Unit (ReLU) for its efficiency in training deep networks. Additionally, sigmoid and tanh activations may be incorporated for specific layers where output constraints or feature transformations benefit from bounded activation.

Computing Environment: The model is designed for deployment in a cloud-based or local computing environment, depending on scalability needs.

Sensors and Control Elements: The invention does not include physical sensors or control mechanisms. Instead, it relies on external biometric measurements and lifestyle/health/demographic data.

Input: The data that is input comes from their most recent blood draw results and from a self-reported survey. Blood draw data consists of various biomarkers, and survey data consists of various lifestyle and health factors. Some biomarkers are engineered into features in a distinct way mentioned before: by using ratios of these biomarkers in relation to one another rather than the biomarkers individually.

Training Data: The system is trained on a multi-site, longitudinal, epidemiologic dataset containing biometric, health, demographic, and lifestyle data. This data is representative of what the input to the system looks like in practice. This data is split into training and testing sets, with k-fold cross-validation being used to validate the training data before performing final evaluations on the test set. The train/test split is 80/20, and k is set to 5.

The training data is representative of the data that is collected in practice. It comes from the SWAN study mentioned earlier, and contains biometric, health, and lifestyle data. The training process minimizes the negative log partial likelihood, a standard loss function for survival models. This loss function ensures that the predicted risk scores maximize the probability of correctly ranking individuals by their time-to-menopause.

The entire modified Cox proportional hazards model, including the feedforward neural network, is trained jointly. There is no separate training phase for the Cox component; the neural network replaces the linear part of the Cox model and learns the log-risk function directly. The baseline survival function is not learned during model training. It is estimated after training using the predicted risk scores on the training data.

In the first phase, the modified Cox proportional hazards model is trained using the training data and the aforementioned methodologies. With the log risk scores that this outputs, a baseline survival function is fit using the Breslow estimator, based on the risk scores produced by the modified Cox proportional hazards model, so that time to menopause can be predicted.

Data Collection: Various biometrics are measured via a blood draw performed by a medical professional. The various lifestyle, demographic, and health factors are determined by a self-reported study.

Pre-processing: The data, both in training and in practice, undergoes various processing steps before being input into the system. The data is standardized using z-score normalization. To account for incomplete observations within the training set, Kaplan-Meier imputation is used. Categorical data is dummy encoded. A core inventive component is the design of a minimal-input architecture, allowing for high-accuracy prediction from a single, phase-adjusted blood draw. This is made possible by engineered features such as the FSH-to-Estradiol ratio and contextual metadata on menstrual cycle phase. These enhancements enable the model to bypass traditional requirements for repeated sampling or full hormonal panels, dramatically expanding its applicability in both clinical and consumer health settings.

Post-processing: The model outputs a personalized log-risk score, which is subsequently translated into an individualized time-to-menopause estimate using a baseline survival function derived from training data via the Breslow estimator. This two-step post-processing pipeline is standard in survival modeling; however, the invention introduces a novel layer of clinical utility by converting these estimates into actionable, user-facing outputs (e.g., expected years to menopause, percentile ranking among population cohort). This interpretability layer is tied directly to the domain-specific risk model, and forms a key differentiator from conventional survival tools which typically remain in abstract risk space.

Learning Categories: The model is trained using a supervised learning paradigm informed by ground-truth menopause transition data from longitudinal cohort studies. While supervised learning is standard, the novelty lies in the application of this approach to a highly censored, sparse, and hormonally variable dataset, coupled with biologically driven feature design. The model is uniquely tailored to learn from these domain constraints, distinguishing it from generic survival prediction systems.

Loss Functions: A loss function of negative log partial likelihood is used during training. The negative log partial likelihood quantifies how well the model predicts the relative risk of experiencing menopause at different times, given the features of individuals. More specifically, it performs the task of maximizing the likelihood that, at each observed event time (i.e., when someone reaches menopause), the model assigns a higher risk score to that individual compared to those who haven't yet experienced menopause. By minimizing the negative log partial likelihood, the model learns to: correctly rank individuals by their risk of reaching menopause at any given time, account for censoring (i.e., individuals for whom menopause hasn't occurred yet during the observation period), and avoid making assumptions about the baseline hazard rate (since it's a partial likelihood).

Optimization Algorithms: The Adam optimizer, otherwise known as the Adaptive Moment Estimation algorithm, is used to efficiently update the neural network's weights during training by adaptively adjusting the learning rate based on the first and second moments of the gradients.

Hyperparameters: During the training process, various hyperparameters are finetuned and experimented with. These include the number of layers in the feedforward neural network, number of nodes per layer, number of epochs (iterations of the Adam optimizer), learning rate of the Adam optimizer, early stopping patience during training, the L2 regularization term within the neural network, the activation functions within the neural network, and the dropout rate used during training to prevent overfitting.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

The present invention may comprise or implement a neural network for machine learning tasks. The neural network may be stored, trained, and/or executed entirely on the user's computer, partly on the user's computer, as a stand-alone software package, partly on the user's computer and partly on a remote computer or entirely on the remote computer or server. The neural network may be stored in the form of program code, as described above. The neural network, in some embodiments, may be a perceptron neural network, a feed forward neural network, a multilayer perceptron neural network, a convolutional neural network, a radial basis functional neural network, a recurrent neural network, a long short-term memory neural network, a sequence-to-sequence neural network model, a modular neural network, or the like.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A computer system (100) for predicting a time to a final menstrual period (FMP) in a patient, the system (100) comprising:
a) a processor (110) configured to execute computer-readable instructions; and
b) a memory component (120) operatively coupled to the processor (110), comprising:
i) an artificial intelligence (AI) model (121), trained by a training data set comprising relationships between a time to FMP and ratios between follicle-stimulating hormone (FSH) levels and estradiol levels, configured to accept FSH data and estradiol levels from a user as input and generate a prediction of a time to FMP as output; and
ii) computer-readable instructions for:
A) receiving a FSH level measurement and an estradiol measurement from the patient;
B) inputting the FSH level measurement and the estradiol measurement into the AI model (121); and
C) receiving the prediction of the time to FMP for the patient from the AI model (121).

2. The system (100) of claim 1, wherein the training data set further comprises relationships between time to FMP and geographical location, age, race, cholesterol, medications, or a combination thereof, wherein the AI model (121) is further configured to accept geographical location, age, race, cholesterol, medications, or a combination thereof from the patient as input.

3. The system (100) of claim 1, wherein the FSH level measurement and the estradiol measurement are derived from a single blood sample from the patient.

4. The system (100) of claim 1, wherein the system (100) is implemented on a local computer, a cloud computing system, or a combination thereof.

5. The system (100) of claim 1, wherein the training data set is standardized by a z-score normalization process.

6. The system (100) of claim 1, wherein the training data set comprises incomplete data supplemented by a Kaplan-Meier estimation.

7. A computer system (100) for predicting a time to a final menstrual period (FMP) in a patient (200), the system (100) comprising:
a) a processor (110) configured to execute computer-readable instructions; and
b) a memory component (120) operatively coupled to the processor (110), comprising:
i) an artificial intelligence (AI) model (121), trained by a training data set comprising relationships between a time to FMP and ratios between follicle-stimulating hormone (FSH) levels and estradiol levels, configured to accept FSH data and estradiol levels from a user as input and generate a prediction of a time to FMP as output; and
ii) computer-readable instructions for:
A) receiving a FSH level measurement and an estradiol measurement from the patient (200);
B) receiving menstrual cycle data from the patient (200);
C) adjusting the FSH level measurement, the estradiol measurement, or a combination thereof based on the menstrual cycle data;
D) inputting the adjusted FSH level measurement and the estradiol measurement into the AI model (121); and
E) receiving the prediction of the time to FMP for the patient (200) from the AI model (121).

8. The system (100) of claim 7, wherein the training data set further comprises relationships between time to FMP and geographical location, age, race, cholesterol, medications, or a combination thereof, wherein the AI model (121) is further configured to accept geographical location, age, race, cholesterol, medications, or a combination thereof from the patient (200) as input.

9. The system (100) of claim 7, wherein the FSH level measurement and the estradiol measurement are derived from a single blood sample from the patient (200).

10. The system (100) of claim 7, wherein the system (100) is implemented on a local computer, a cloud computing system, or a combination thereof.

11. The system (100) of claim 7, wherein the training data set is standardized by a z-score normalization process.

12. The system (100) of claim 7, wherein the training data set comprises incomplete data supplemented by a Kaplan-Meier estimation.

13. A computer-implemented method for predicting a time to a final menstrual period (FMP) in a patient (200), the method comprising:
a) providing an artificial intelligence (AI) model, trained by a training data set comprising relationships between a time to FMP and ratios between follicle-stimulating hormone (FSH) levels and estradiol levels, configured to accept FSH data and estradiol levels from a user as input and generate a prediction of a time to FMP as output;
b) receiving a FSH level measurement and an estradiol measurement from the patient (200);
c) inputting the FSH level measurement and the estradiol measurement into the AI model; and
d) receiving the prediction of the time to FMP for the patient (200) from the AI model.

14. The method of claim 13 further comprising:
a) receiving menstrual cycle data from the patient (200); and
b) adjusting the FSH level measurement, the estradiol measurement, or a combination thereof based on the menstrual cycle data.

15. The method of claim 13, wherein the training data set further comprises relationships between time to FMP and geographical location, age, race, cholesterol, medications, or a combination thereof, wherein the AI model is further configured to accept geographical location, age, race, cholesterol, medications, or a combination thereof from the patient (200) as input.

16. The method of claim 13 further comprising:
a) receiving a single blood sample from the patient (200); and
b) deriving the FSH level measurement and the estradiol measurement from the single blood sample.

17. The method of claim 13, wherein the method is implemented on a system (100) comprising a local computer, a cloud computing system, or a combination thereof.

18. The method of claim 13, wherein the training data set is standardized by a z-score normalization process.

19. The method of claim 13, wherein the training data set comprises incomplete data supplemented by a Kaplan-Meier estimation.
